(19) United States
(12) Patent Application Publication
(10) Pub. No.: US 2021/0146362 A1
(43) Pub. Date: May 20, 2021

(54) BLOOD COAGULATION ANALYSIS DEVICE AND METHOD OF CLEANING DISPENSATION NOZZLE

(71) Applicant: SHIMADZU CORPORATION, Kyoto-shi (JP)

(72) Inventors: Junichi MATSUMOTO, Kyoto-shi (JP); Kiyohiro SUGIYAMA, Kyoto-shi (JP); Takanori ONOKI, Kyoto-shi (JP)

(73) Assignee: SHIMADZU CORPORATION, Kyoto-shi (JP)

(21) Appl. No.: 17/094,946

(22) Filed: Nov. 11, 2020

(30) Foreign Application Priority Data
Nov. 14, 2019 (JP) 2019-206468

(51) Int. Cl. B01L 3/00; G01N 33/86

(52) U.S. Cl. CPC B01L 3/502761; B01L 2400/021; G01N 33/86

(57) ABSTRACT

A cleaning condition of a first nozzle can be set for each reagent combination in a controller of a blood coagulation analysis device, the reagent combination indicating a combination of a reagent previously dispensed by the first nozzle and a reagent currently dispensed by the first nozzle. When the controller of the blood coagulation analysis device obtains the reagent combination and the cleaning condition of the first nozzle is set for the obtained reagent combination, the controller performs control to clean the first nozzle under the set cleaning condition.

### BACKGROUND OF THE INVENTION

### Field of the Invention

The present disclosure relates to a blood coagulation analysis device and a method of cleaning a dispensation nozzle.

### Description of the Background Art

Japanese Patent Laying-Open No. 2017-96760 discloses an automatic analysis device that dispenses each of a specimen and a reagent into a reaction container and that optically measures changes in color tone and turbidity caused by a reaction between the specimen and the reagent. The reaction container having been through the measurement is cleaned after the measurement and is used for next measurement.

### SUMMARY OF THE INVENTION

In the automatic analysis device described in Japanese Patent Laying-Open No. 2017-96760, after performing the dispensation, a nozzle (also referred to as a “probe” in the blood coagulation analysis device) used for the dispensation is cleaned in a cleaning bath. In such a method, it takes time and effort to manage the cleaning bath. Further, when the nozzle is cleaned always in the same manner regardless of a state of the nozzle after the dispensation, the nozzle may not be sufficiently cleaned. When the nozzle is not sufficiently cleaned, accuracy of analysis may be decreased due to an influence of a foreign matter attached to the nozzle. For example, when a nozzle having dispensed (previously dispensed) a reagent is not sufficiently cleaned and another reagent is dispensed (currently dispensed) using the nozzle, contamination (hereinafter, also referred to as “reagent-to-reagent contamination”) resulting from the previously dispensed reagent may occur. The reagent-to-reagent contamination leads to decreased accuracy of analysis.

The present disclosure has been made to solve the above-described problem and has an object to provide a blood coagulation analysis device and a method of cleaning a dispensation nozzle, so as to suppress reagent-to-reagent contamination.

A blood coagulation analysis device according to a first aspect of the present disclosure performs an analysis by reacting a specimen and a reagent in a reaction container, and includes a reagent port, a first dispensation device, and a controller. At the reagent port, a plurality of types of reagents are able to be provided. The first dispensation device has a first nozzle and a first driving device. The first driving device moves the first nozzle to change a position of the first nozzle. The first dispensation device suctions a predetermined reagent of the plurality of types of reagents at the reagent port using the first nozzle, and dispenses the suctioned reagent from the first nozzle to the reaction container. The controller controls the first dispensation device. The controller controls the first dispensation device to dispense the reagent using the first nozzle and to clean the first nozzle whenever the dispensation of reagent is performed. A cleaning condition of the first nozzle is able to be set for each reagent combination in the controller, the reagent combination indicating a combination of a reagent previously dispensed by the first nozzle and a reagent currently dispensed by the first nozzle. When the controller obtains the reagent combination and the cleaning condition of the first nozzle is set for the obtained reagent combination, the controller controls the first dispensation device to clean the first nozzle under the set cleaning condition.

A method of cleaning a dispensation nozzle according to a second aspect of the present disclosure is a method of cleaning a dispensation nozzle that dispenses a reagent in a blood coagulation analysis device for performing an analysis by reacting a specimen and the reagent in a reaction container, and includes the following first to third steps.

In the first step, a controller of the blood coagulation analysis device obtains a reagent combination indicating a combination of a reagent previously dispensed by the dispensation nozzle and a reagent currently dispensed by the dispensation nozzle.

In the second step, the controller determines whether or not a cleaning condition of the dispensation nozzle is set for the reagent combination obtained in the first step.

In the third step, when it is determined in the second step that the cleaning condition of the dispensation nozzle is set for the reagent combination, the controller performs control to clean the dispensation nozzle under the cleaning condition set for the reagent combination.

It should be noted that the controller of the blood coagulation analysis device may be constituted of a single unit or may be constituted of a plurality of divided units.

The foregoing and other objects, features, aspects and advantages of the present invention will become more apparent from the following detailed description of the present invention when taken in conjunction with the accompanying figures.

### BRIEF DESCRIPTION OF THE FIGURES

FIG. 1 is a diagram showing a configuration for transferring and discarding a reaction container and stirring and measuring a content of a reaction container in a blood coagulation analysis device according to an embodiment of the present disclosure.

FIG. 2 is a diagram showing configurations of first and second dispensation devices and devices therearound in the blood coagulation analysis device according to the embodiment of the present disclosure.

FIG. 3 is a plan view of an analysis table included in the blood coagulation analysis device according to the embodiment of the present disclosure.

FIG. 4 is a diagram for illustrating a structure of each arm shown in FIG. 3.

FIG. 5 is a diagram showing a control system of the blood coagulation analysis device according to the embodiment of the present disclosure.

FIG. 6 is a diagram showing exemplary probe cleaning information.

FIG. 7 is a flowchart showing a series of flows of an analysis performed by the blood coagulation analysis device according to the embodiment of the present disclosure.

FIG. 8 is a flowchart showing control associated with cleaning of each of the first probe and the second probe performed during execution of the analysis shown in FIG. 7.

FIG. 9 is a flowchart showing a modification of the control associated with the cleaning of the probes as shown in FIG. 8.

FIG. 10 is a diagram showing an exemplary cleaning condition input screen.

### DESCRIPTION OF THE PREFERRED EMBODIMENTS

Hereinafter, embodiments of the present disclosure will be described in detail with reference to the figures. It should be noted that in the figures, the same or corresponding portions are denoted by the same reference characters, and will not be described repeatedly.

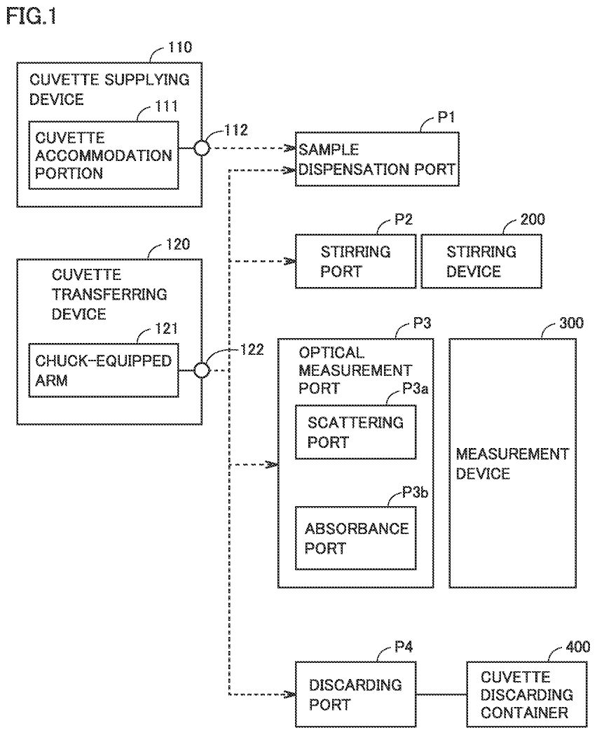

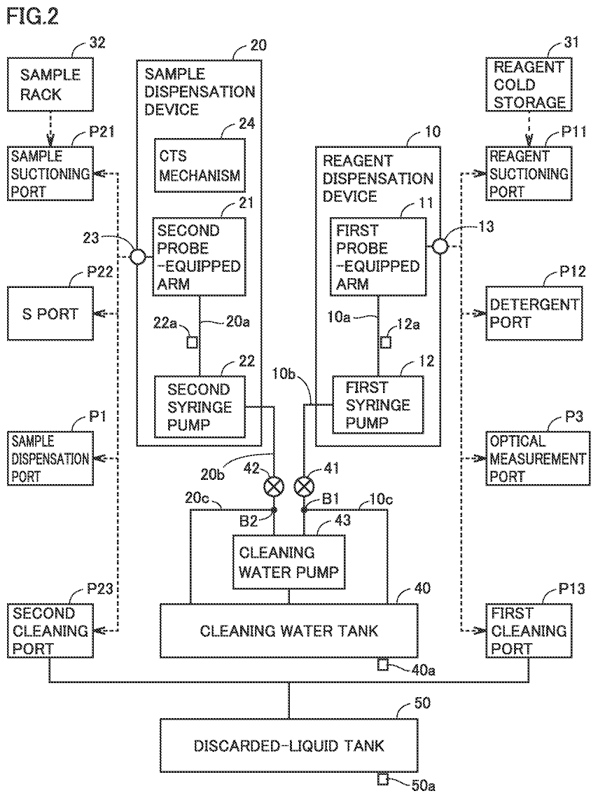

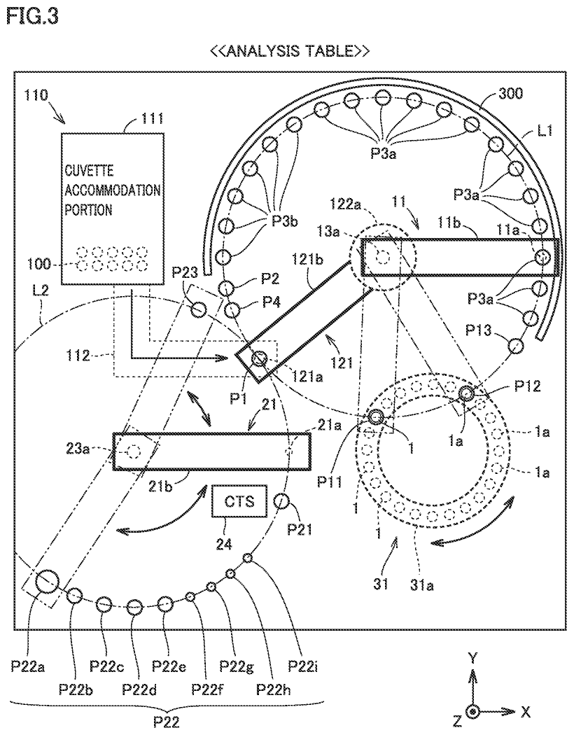

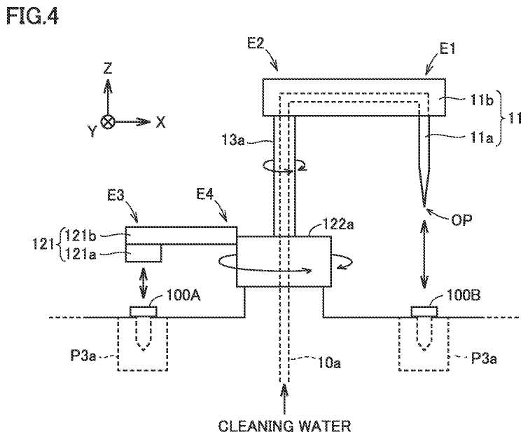

A blood coagulation analysis device according to the present embodiment (hereinafter, also simply referred to as “analysis device”) dispenses each of a specimen and a reagent into a reaction container using a dispensation nozzle and optically measures a reaction state in the reaction container. In the description below, the dispensation nozzle and the specimen will be referred to as “probe” and “sample”, respectively. Examples of the sample employed herein include a blood component and urine. In the present embodiment, as the reaction container of the analysis device, a disposable cuvette (for example, below-described cuvettes 100, 100A, 100B shown in FIGS. 3 and 4) is employed. Hereinafter, a configuration of the analysis device will be described with reference to FIGS. 1 and 2.

FIG. 1 is a diagram showing a configuration for transferring and discarding a reaction container and stirring and measuring a content of the reaction container in the analysis device.

Referring to FIG. 1, the analysis device includes a cuvette supplying device 110, a cuvette transferring device 120, a stirring device 200, a measurement device 300, and a cuvette discarding container 400. Cuvette supplying device 110 and cuvette transferring device 120 according to the present embodiment correspond to examples of the “supplying device” and the “transferring device” according to the present disclosure, respectively. Hereinafter, cuvette supplying device 110 and cuvette transferring device 120 will be simply referred to as “supplying device 110” and “transferring device 120”, respectively.

The analysis device further includes a sample dispensation port P1. Supplying device 110 includes a cuvette accommodation portion 111 (hereinafter, simply referred to as “accommodation portion 111”) and a supplying mechanism 112. Accommodation portion 111 can accommodate a plurality of cuvettes (for example, 1000 cuvettes at maximum). Supplying mechanism 112 supplies the cuvette accommodated in accommodation portion 111 to sample dispensation port P1. Details of accommodation portion 111 and supplying mechanism 112 will be described later (sec FIG. 3).

Sample dispensation port P1 (hereinafter, also simply referred to as “port P1”) is disposed at a position at which a sample can be dispensed by a below-described sample dispensation device 20 (see FIG. 2). When the cuvette is set at port P1, a sample is dispensed into a cuvette by sample dispensation device 20.

Transferring device 120 includes a chuck-equipped arm 121 (hereinafter, simply referred to as “arm 121”) and a driving device 122. Arm 121 has a chuck that grasps a cuvette. Arm 121 detachably holds a cuvette using the chuck. Driving device 122 moves arm 121 (thus, the chuck) to change a position of the chuck. Details of arm 121 and driving device 122 will be described later (see FIGS. 3 and 4).

The analysis device further includes a plurality of ports to which cuvettes can be transferred by transferring device 120, more specifically, a stirring port P2, a scattering port P3a, an absorbance port P3b, and a discarding port P4. Each of sample dispensation port P1, stirring port P2, scattering port P3a, absorbance port P3b, and discarding port P4 is provided with a sensor (hereinafter, also referred to as “port sensor”) that detects presence or absence of a cuvette.

Stirring port P2 is disposed at a stirring position of stirring device 200. When a cuvette is set at stirring port P2, stirring device 200 stirs the content of the cuvette under a predetermined condition (for example, stirring speed and stirring time).

Each of scattering port P3a and absorbance port P3b is disposed at a measurement position of measurement device 300. Hereinafter, unless stated distinctively, each of scattering port P3a and absorbance port P3b will be referred to as “optical measurement port P3”.

Measurement device 300 performs a predetermined measurement onto the content of the cuvette. in the present embodiment, measurement device 300 has a light source and a photodetector (each not shown), and emits light of the light source onto the content of the cuvette set at one of optical measurement ports P3 to measure a reaction state in the cuvette based on a change in an amount of light detected by the photodetector. Measurement device 300 includes a light source and a photodetector for scattering port P3a and a light source and a photodetector for absorbance port P3b. As the light source and the photodetector for scattering port P3a, a light emitting diode and a photodiode can be employed, respectively. The photodetector for scattering port P3a is disposed to detect 90°-scattered light (i.e., light scattered in a direction orthogonal to a light emission direction). As the light source and the photodetector for absorbance port P3b, a halogen lamp and a photodiode can be employed, respectively. Measurement device 300 may have a plurality of halogen lamps with different wavelengths, and may select and use one halogen lamp suitable for the measurement among the halogen lamps. The photodetector for absorbance port P3b is disposed to detect an amount of light having passed therethrough.

A used cuvette is to be collected at discarding port P4. Discarding port P4 is connected to a cuvette discarding container 400 (hereinafter, simply referred to as “discarding container 400”) via a pipe, for example. When a cuvette is introduced into discarding port P4, the cuvette is guided to discarding container 400.

FIG. 2 is a diagram showing configurations of a reagent dispensation device 10, a sample dispensation device 20, and devices therearound in the analysis device.

Referring to FIG. 2, the analysis device includes reagent dispensation device 10, sample dispensation device 20, a reagent cold storage 31, a sample rack 32, a cleaning water tank 40, a monitoring unit 40a, a first switching valve 41, a second switching valve 42, a cleaning water pump 43, a discarded-liquid tank 50, and a monitoring unit 50a. Reagent dispensation device 10 and sample dispensation device 20 according to the present embodiment correspond to examples of the “first dispensation device” and the “second dispensation device” according to the present disclosure, respectively. Hereinafter, reagent dispensation device 10 and sample dispensation device 20 will be simply referred to as “dispensation device 10” and “dispensation device 20”, respectively.

A reagent tray is accommodated in reagent cold storage 31. A plurality of reagent containers are set on the reagent tray. The plurality of reagent containers hold respective different ones or the plurality of types of reagents. Reagent cold storage 31 has: a temperature regulation device (not shown) that regulates a temperature in the cold storage; and a temperature sensor (not shown) that detects the temperature in the cold storage. Reagent cold storage 31 cools the reagent tray (thus, the reagents). Reagent cold storage 31 may further include a stirring device that stirs a reagent in each reagent container set on the reagent tray.

Dispensation device 10 includes a first probe-equipped arm 11 (hereinafter, simply referred to as “arm 11”), a first syringe pump 12, a pressure sensor 12a, and a first driving device 13. Arm 11 has a first probe (first nozzle) at the leading end of the arm main body. Arm 11 has: a temperature regulation device (not shown) that regulates a temperature of the first probe; and a temperature sensor (not shown) that detects the temperature of the first probe. Arm 11 maintains the temperature of the first probe at a predetermined temperature. First driving device 13 moves arm 11 (thus, the first probe) to change a position of the first probe. Details of arm 11 and first driving device 13 will be described later (see FIGS. 3 and 4).

The analysis device further includes ports to each of which the first probe can be moved by first driving device 13, more particularly, a reagent suctioning port P11, a detergent port P12, and a first cleaning port P13. Further, first driving device 13 can move the first probe to optical measurement ports P3 (i.e., scattering port P3a and absorbance port P3b shown in FIG. 1) described above.

At reagent suctioning port P11 (hereinafter, also simply referred to as “port P11”), a plurality of types of reagents can be provided. Port P11 in the present embodiment is an opening located above reagent cold storage 31 to guide the first probe to a reagent. The analysis device according to the present embodiment has one port P11 and changes a reagent to be located immediately below port P11. Although details will be described later, reagent cold storage 31 has a turntable (not shown) for rotating the reagent tray. When the turntable is rotated, the position of the reagent container is changed. When the first probe is moved to port P11, a predetermined reagent container of the plurality of reagent containers set on the reagent tray is placed immediately below port P11. The first probe can suction the reagent in the reagent container through port P11. Port P11 according to the present embodiment corresponds to an example of the “reagent port” according to the present disclosure. It should be noted that the analysis device may have a plurality of respective reagent ports for the reagents. By preparing the respective reagents at the reagent ports, the plurality of types of reagents can be provided at the plurality of reagent ports.

At detergent port P12, a detergent can be provided. Detergent port P12 in the present embodiment is an opening for guiding the first probe to the detergent. The detergent is prepared immediately below detergent port P12. The first probe can suction the detergent through detergent port P12. The detergent may be always present immediately below detergent port P12, or a predetermined detergent may be transferred to immediately below detergent port P12 when the first probe is moved to detergent port P12. Although details will be described later, in the present embodiment, detergent port P12 is located above reagent cold storage 31, and the detergent container is set on the reagent tray. The detergent container is transferred to immediately below detergent port P12 by the turntable of reagent cold storage 31. Although reagent suctioning port P11 and detergent port P12 are shown to be located at different positions in FIG. 3, the reagent suctioning port and the detergent port may be disposed at the same position. That is, one port (for example, port P11 or detergent port P12) may function as both the reagent suctioning port and the detergent port. Since the reagent container and the detergent container are set on the reagent tray, any one of the reagent container and the detergent container can be moved to immediately below the port by rotating the turntable.

A used cleaning liquid (for example, pure water and detergent) is to be collected at first cleaning port P13. First cleaning port P13 in the present embodiment is an opening for guiding the first probe to discarded-liquid tank 50. First cleaning port P13 is connected to discarded-liquid tank 50 via a pipe, for example. The cleaning liquid discharged from the first probe to first cleaning port P13 is accumulated in discarded-liquid tank 50. Monitoring unit 50a includes: a liquid level sensor (not shown) that detects a liquid level in discarded-liquid tank 50; and a notification device (not shown) (for example, a lamp and an alarm). When the liquid level in discarded-liquid tank 50 exceeds a predetermined value, monitoring unit 50a notifies, by way of a sound and/or presentation, that the amount of discarded-liquid has become large.

Details of reagent suctioning port P11, detergent port P12, and first cleaning port P13 will be described later (see FIG. 3).

First syringe pump 12 is located between cleaning water pump 43 and arm 11 (including the first probe). The first probe of arm 11 and first syringe pump 12 are connected to each other via a pipe 10a. Pipe 10a is connected to the first probe through the inside of the arm main body (see, for example, FIG. 4 described later). A pressure sensor 12a is provided in pipe 10a. First syringe pump 12 includes: a cylinder (not shown) having a cylindrical shape; a plunger (not shown) provided to slide on an inner wall surface of the cylinder so as to move in a reciprocating manner (move up and down) in the cylinder; and an actuator (not shown) (for example, an electric motor) that drives the plunger. In accordance with control of driving of the plunger, first syringe pump 12 can adjust suctioning pressure and discharging pressure of the first probe. A pressure of the first probe is detected by pressure sensor 12a.

Cleaning water tank 40 accommodates pure water (for example, purified water). Monitoring unit 40a includes: a water level sensor (not shown) that detects a water level in cleaning water tank 40; and a notification device (not shown) (for example, a lamp and an alarm). When the water level in cleaning water tank 40 falls below a predetermined value, monitoring unit 40a notifies, by way of a sound and/or presentation, that the amount of water has become small.

Cleaning water pump 43 pumps up the pure water from cleaning water tank 40. Cleaning water pump 43 supplies the pure water accommodated in cleaning water tank 40 to first syringe pump 12 and arm 11 (thus, the first probe). Cleaning water pump 43 is connected to first syringe pump 12 via a pipe 10b. First switching valve 41 (for example, an electromagnetic valve) is provided in pipe 10b. Pipe 10b has a branch portion B1 at a position on the cleaning water pump 43 side with respect to first switching valve 41. A pipe 10c, which is connected to cleaning water tank 40, is connected to branch portion B1. Pipe 10c functions as a pipe (so-called return pipe) for returning, to cleaning water tank 40, the water pumped up by cleaning water pump 43. It should be noted that each of pipes 10a to 10c may be a tube composed of a resin.

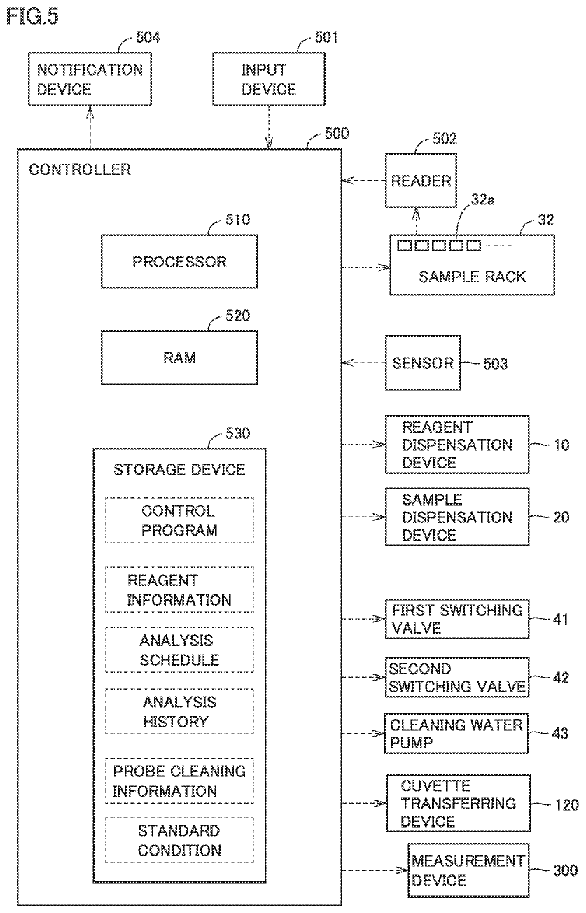

Before performing dispensation by dispensation device 10, a controller of the analysis device (for example, a controller 500 shown in FIG. 5 described later) operates cleaning water pump 43 to bring first switching valve 41 into an open state. Thus, the pure water in cleaning water tank 40 is supplied to arm 11 through pipe 10b, first syringe pump 12, and pipe 10a. Then, the controller performs control to close first switching valve 41 with the pure water being introduced from first switching valve 41 up to the leading end of arm 11 (i.e., the opening of the first probe). Even after first switching valve 41 is closed, the controller does not stop cleaning water pump 43, and performs control to circulate the water pumped up by cleaning water pump 43, by returning the water to cleaning water tank 40 through pipe 10c. The dispensation by dispensation device 10 is performed in this state.

In order to perform suctioning using the first probe, the controller moves down the plunger of first syringe pump 12 (i.e., moves the plunger in a direction in which the cylinder volume is increased). Accordingly, suctioning pressure is generated at the opening of the first probe, thereby performing suctioning using the first probe. In order to perform discharging using the first probe, the controller moves up the plunger of first syringe pump 12 (i.e., moves the plunger in a direction in which the cylinder volume is decreased). Accordingly, discharging pressure is generated at the opening of the first probe, thereby performing discharging using the first probe. First syringe pump 12 applies, to the first probe via the water introduced as described above, a pressure (i.e., the suctioning pressure and discharging pressure) generated in response to a change in cylinder volume. It should be noted that when the water introduced in the first probe is brought into contact with the reagent suctioned from the first probe, the reagent may be diluted. In order to suppress such dilution of the reagent, dispensation device 10 may suction a small amount of air using the first probe before suctioning the reagent using the first probe. By forming a gap (for example, air gap) between the water and the reagent, the dilution of the reagent can be suppressed.

Dispensation device 20 includes a second probe-equipped arm 21 (hereinafter, simply referred to as “arm 21”), a second syringe pump 22, a pressure sensor 22a, and a second driving device 23. Arm 21 has a second probe (second nozzle) at the leading end of the arm main body. Second driving device 23 moves arm 21 (thus, the second probe) to change a position of the second probe. The second probe of arm 21 and second syringe pump 22 are connected to each other via a pipe 20a. Pressure sensor 22a is provided in pipe 20a. Second syringe pump 22 is connected to cleaning water pump 43 via a pipe 20b. Second switching valve 42 (for example, an electromagnetic valve) is provided in pipe 20b. Pipe 20b has a branch portion B2 at a position on the cleaning water pump 43 side with respect to second switching valve 42. A pipe 20c, which is connected to cleaning water tank 40, is connected to branch portion B2.

Arm 21, second syringe pump 22, pressure sensor 22a, second driving device 23, pipes 20a to 20c, branch portion B2, and second switching valve 42 have the same structures as those of arm 11, first syringe pump 12, pressure sensor 12a, first driving device 13, pipes 10a to 10c, branch portion B1, and first switching valve 41, respectively. Hence, they will not be described in detail.

Dispensation device 20 further includes a CTS (Closed Tube Sampling) mechanism 24. CTS mechanism 24 pierces a cap using a piercer when the sample container described later is provided with the cap. When the cap of the sample container is pierced, the sample in the sample container can be suctioned by the second probe.

The analysis device further includes ports to each of which the second probe can be moved by second driving device 23, more specifically, a sample suctioning port P21, an S port P22, and a second cleaning port P23. Second driving device 23 can also move the second probe to sample dispensation port P1 (FIG. 1) described above.

A plurality of sample containers are set on sample rack 32. The plurality of sample containers may include, for example, a sample container that holds a blood component (for example, plasma) and a sample container that holds urine. Each of the blood component and the urine corresponds to a sample. At sample suctioning port P21 (hereinafter, also simply referred to as “port P21”), a plurality of types of samples can be provided. Port P21 in the present embodiment is an opening located above sample rack 32 to guide the second probe to a sample. The analysis device according to the present embodiment has one port P21, and change a sample to be located immediately below port P21. Although details will be described later, sample rack 32 is movable and can change a position of the sample container. When the second probe is moved to port P21, a predetermined sample container of the plurality of sample containers set on sample rack 32 is placed at a position immediately below port P21. The second probe can suction the sample in the sample container through port P21. Port P21 according to the present embodiment corresponds to an example of the “specimen port” according to the present disclosure. It should be noted that the analysis device may have a plurality of respective specimen ports for specimens. By preparing the respective specimens at the specimen ports, the plurality of types of specimens can be provided at the plurality of specimen ports.

S port P22 includes a plurality of ports. In the present embodiment, S port P22 includes: a plurality of detergent ports at which a plurality of types of detergents are to be provided; a plurality of diluent ports at which a plurality of types of sample diluents (hereinafter, also simply referred to as “diluents”) are to be provided; and a plurality of buffer ports at which a plurality of types of sample butlers (hereinafter, also simply referred to as “buffers”) are to be provided. A detergent is prepared immediately below each detergent port, a diluent is prepared immediately below each diluent port, and a buffer is prepared immediately below each butler port. The second probe can respectively suction the detergent, the diluent, and the buffer through the detergent port, diluent port, and buffer port included in S port P22.

At second cleaning port P23, used cleaning liquid (for example, pure water and detergent) is to be collected. Second cleaning port P23 in the present embodiment is an opening for guiding the second probe to discarded-liquid tank 50. Second cleaning port P23 is connected to discarded-liquid tank 50 via a pipe, for example. The cleaning liquid discharged from the second probe to second cleaning port P23 is accumulated in discarded-liquid tank 50.

Details of sample suctioning port P21, S port P22, and second cleaning port P23 will be described later (see FIG. 3).

FIG. 3 is a plan view of an analysis table included in the analysis device according to the present embodiment. In FIG. 3, three axes (X axis, Y axis, and Z axis) orthogonal to one another are shown. Among the X axis, the Y axis, and the Z axis, the X axis indicates the width direction of the analysis device, the Y axis indicates the depth direction of the analysis device, and the Z axis indicates the vertical direction (i.e., upward/downward direction). A direction indicated by a Z-axis arrow corresponds to the “upward direction” and a direction opposite thereto corresponds to the “downward direction (i.e., gravity direction)”.

Referring to FIG. 3 together with FIGS. 1 and 2, accommodation portion 111 accommodates a plurality of (for example, 500) cuvettes 100. A user can supply a cuvette 100 from an introduction opening (not shown) of accommodation portion 111 into accommodation portion 111. Although any material is usable for cuvette 100, a cuvette 100 composed of acrylic is employed in the present embodiment. Supplying mechanism 112 takes out cuvettes 100 from accommodation portion 111 and supply cuvettes 100 to port P1 one by one. Any method is usable to transfer cuvettes 100 in supplying mechanism 112. For example, any one of a slope method (a self-weight method), a belt conveyor method, a roller method, and a slide method may be used. Supplying mechanism 112 receives a detection result of the port sensor of port P1, and supplies next cuvette 100 to port P1 when port P1 becomes vacant. However, it is not limited thereto. Supplying mechanism 112 may supply cuvette 100 to port P1 in accordance with an instruction of controller 500 (see FIG. 5) described later.

Arm 21 includes a second probe 21a and an arm main body 21b. Second driving device 23 (FIG. 2) includes: a rotation shaft 23a connected to arm main body 21b; a rotation actuator (not shown) that rotates rotation shall 23a; and an ascending/descending actuator (not shown) that displaces arm 21 upward/downward. When rotation shalt 23a is rotated, arm 21 (thus, second probe 21a) is rotated together with rotation shaft 23a. With rotation shaft 23a rotated, second probe 21a is rotated around rotation shaft 23a to move on an are-shaped trajectory L2 in the XY plane. Second driving device 23 can move second probe 21a to each of port. P1, port P21, S port P22 (more specifically, ports P22a to P22i), and second cleaning port P23 provided on trajectory L2. In S port P22, ports P22a, P22b are detergent ports, ports P22c, P22d, P22e are buffer/diluent ports, ports P22f, P22g are diluent/normal plasma/deficient plasma ports, and ports P22h, P22i are normal plasma/deficient plasma ports.

Although not shown in FIG. 3, sample rack 32 (FIG. 2) loaded on the analysis device (rack-loaded) is located below port P21. Sample rack 32 movably holds the sample containers. Prior to dispensing a sample, sample rack 32 places, immediately below port P21, a target sample container for the dispensation. In sample rack 32, a mechanism that moves the target sample container immediately below port P21 may be of a rotation type (for example, a turntable type) or of a slide type. CTS mechanism 24 is located in the vicinity of port P21, and pierces a cap of the target sample container using a piercer when the target sample container is provided with the cap.

Reagent cold storage 31 is located below port P11 and detergent port P12. A reagent tray 31a having a plurality of reagent containers 1 and a plurality of detergent containers 1a set thereon is accommodated in reagent cold storage 31. The plurality of detergent containers 1a hold different respective types of detergents. Reagent cold storage 31 includes a disc-shaped turntable (not shown) and an actuator (not shown) (for example, an electric motor) that rotates the turntable. The reagent tray is fixed (for example, fixed by a latch) on the turntable. When the turntable is rotated, the positions of reagent containers 1 and detergent containers 1a are changed. Each of port P11 and detergent port P12 is located on the rotation trajectory of the turntable. By driving the turntable, reagent cold storage 31 can place a reagent container 1 immediately below port P11 and can place a detergent container 1a immediately below detergent port P12.

Arm 11 includes a first probe 11a and an arm main body 11b. First driving device 13 (FIG. 2) includes: a rotation shaft 13a connected to arm main body 11b; a rotation actuator (not shown) that rotates rotation shaft 13a; and an ascending/descending actuator (not shown) that displaces arm 11 upward/downward. When rotation shaft 13a is rotated, arm 11 (thus, first probe 11a) is rotated together with rotation shaft 13a.

Arm 121 includes a chuck 121a and an arm main body 121b. Chuck 121a holds cuvette 100 in any manner. Chuck 121a may be a mechanical chuck, a magnet chuck, or a vacuum chuck. Driving device 122 (FIG. 1) includes: a rotation shaft 122a connected to arm main body 121b; a rotation actuator (not shown) that rotates rotation shaft 122a; and an ascending/descending actuator (not shown) that displaces arm 121 upward/downward. When rotation shaft 122a is rotated, arm 121 (thus, chuck 121a) is rotated together with rotation shaft 122a.

The respective rotation centers of rotation shaft 13a and rotation shaft 122a are the same. When each of first probe 11a and chuck 121a is driven to be rotated in the above-described manner, each of first probe 11a and chuck 121a is moved on a circular trajectory L1 in the XY plane. First driving device 13 can move first probe 11a to each port provided on trajectory L1. Driving device 122 can move chuck 121a to each port provided on trajectory L1. Port P1, stirring port P2, the plurality of scattering ports P3a, the plurality of absorbance ports P3b, discarding port P4, port P11, detergent port P12, and first cleaning port P13 are provided on trajectory L1.

FIG. 4 is a diagram for illustrating the structures of arm 11 and arm 121 shown in FIG. 3. The X axis, the Y axis, and the Z axis in FIG. 4 correspond to the X axis, the Y axis, and the Z axis in FIG. 3, respectively.

Referring to FIG. 4 together with FIG. 3, arm 11 and arm 121 are disposed to be displaced from each other in the upward/downward direction. In the present embodiment, arm 11 is disposed at a position higher than that of arm 121. First probe 11a is connected to a leading end portion E1 of arm main body 11b, and rotation shaft 13a is connected to a base end portion E2 of arm main body 11b. First probe 11a is provided with an opening OP at its leading end. When the ascending/descending actuator (not shown) of first driving device 13 moves arm 11 and rotation shaft 13a together in the upward/downward direction, arm 11 (thus, first probe 11a) is displaced upward/downward. For example, in order to dispense a reagent into a cuvette 100B shown in FIG. 4 (i.e., a cuvette 100 set at scattering port P3a), first probe 11a is moved down to approach cuvette 100B. When the dispensation of reagent is finished, first probe 11a is moved up and away from cuvette 100B.

As shown in FIG. 4, pipe 10a (see FIG. 2) described above is connected to first probe 11a via pipes provided inside rotation shaft 13a and arm main body 11b. Via the pipes, cleaning water pump 43 (FIG. 2) can supply first probe 11a with pure water (cleaning water) in cleaning water tank 40 (FIG. 2). Further, first syringe pump 12 can apply a discharging pressure to first probe 11a to discharge the liquid in arm 11 (i.e., first probe 11a and arm main body 11b) from opening OP of first probe 11a.

Chuck 121a is connected to a leading end portion E3 of arm main body 121b, and rotation shaft 122a is connected to a base end portion E4 of arm main body 121b. Base end portion E4 of arm main body 121b is held by rotation shaft 122a in such a manner that base end portion E4 is displaceable in the upward/downward direction. When the ascending/descending actuator (not shown) of driving device 122 moves arm 121 in the upward/downward direction, arm 121 (thus, chuck 121a) is displaced upward/downward. For example, in order to transfer a cuvette 100A shown in FIG. 4 (i.e., a cuvette 100 set at scattering port P3a), chuck 121a is moved down to grasp cuvette 100A, and is then moved up and away from scattering port P3a with cuvette 100A being grasped. Thereafter, when arm 121 is driven to be rotated by driving device 122 and chuck 121a reaches a transfer destination port (more particularly, any port located on trajectory L1 shown in FIG. 3), chuck 121a is moved down again to set cuvette 100A at the port. When cuvette 100A is set at the port, chuck 121a releases cuvette 100A (i.e., the chuck is disengaged) and is moved up again.

FIG. 5 is a diagram showing a control system of the analysis device according to the present embodiment. Referring to FIG. 5, the analysis device includes controller 500. Controller 500 includes a processor 510, a RAM (Random Access Memory) 520, a storage device 530, and an input/output buffer (not shown) for inputting and outputting various types of signals. As processor 510, for example, a CPU (Central Processing Unit) can be employed. RAM 520 functions as a working memory for temporarily storing data to be processed by processor 510. Storage device 530 can store written information. Storage device 530 includes a ROM (Read Only Memory) and a rewritable nonvolatile memory, for example. Storage device 530 may include at least one of a hard disk drive and an SSD (Solid State Drive). Control programs to be used for various types of control, and information (for example, various types of parameters) to be used in the programs are stored in storage device 530 in advance. The various types of processes to be performed by controller 500 can be performed by dedicated hardware (electronic circuit) instead of software. Any number of processors may be included in the analysis device and the processor(s) included in the analysis device may be arranged in any manner. A processor may be prepared for each predetermined control or respective processors may be mounted for a plurality of units.

Controller 500 controls dispensation device 10 (for example, first syringe pump 12 and first driving device 13 shown in FIG. 2), dispensation device 20 (for example, second syringe pump 22, second driving device 23, and CTS mechanism 24 shown in FIG. 2), first switching valve 41, second switching valve 42, cleaning water pump 43, transferring device 120 (for example, chuck 121a and driving device 122 shown in FIGS. 1 and 4), and measurement device 300.

The analysis device includes a sensor 503 that detects a state of the analysis device. Output signals of various types of sensors included in sensor 503 (i.e., signals indicating detection results) are input to controller 500. In the present embodiment, sensor 503 includes pressure sensors 12a, 22a (FIG. 2), the temperature sensors (not shown) of the first and second probes, and the port sensors (not shown).

The analysis device further includes an input device 501 and a notification device 504. Input device 501 is a device that receives an input from a user. Input device 501 is manipulated by the user and outputs a signal corresponding to the user's manipulation to controller 500. Notification device 504 performs a predetermined notification process for the user when a request is made from controller 500. In the present embodiment, a touch panel display having input device 501 and notification device 504 integrated therein is employed. However, it is not limited thereto and input device 501 and notification device 504 may be separately prepared. For example, various types of pointing devices (such as a mouse and a touch pad), a keyboard, and the like may be employed as input device 501. Alternatively, input device 501 may be a manipulation portion of a mobile device (for example, a smartphone). Meanwhile, the notification process for the user may be performed in any manner. The notification may be performed by way of presentation on a display device (for example, presentation of letters and characters or presentation of an image), may be performed by way of a sound (inclusive of voice) using a speaker, or may be performed by way of lighting (inclusive of blinking) of a predetermined lamp.

In the present embodiment, each of the sample containers set on sample rack 32 is provided with a tag 32a. The analysis device further includes a reader 502 that reads information from tag 32a. Reader 502 outputs information obtained from tag 32a to controller 500. Tag 32a provides information (for example, a sample ID, a test of analysis, or the like) of the sample in the sample container. Tag 32a presents a predetermined code (for example, QR code (registered trademark)). However, it is not limited thereto and tag 32a may be an IC tag or may transmit sample information using electric wave. A reading method of reader 502 may be a non-contact method or a contact method. A known code reader may be employed as reader 502 that reads the code presented on tag 32a. Reader 502 may be fixed to a position at which tag 32a can be read, or may be a movable reader controlled by controller 500.

Information (for example, a reagent ID, a type of reagent, an expiry date, or the like) of each reagent in reagent cold storage 31 (FIG. 2) is registered in controller 500. The reagent information may be registered in controller 500 by the user. However, it is not limited thereto. A tag may be provided for each reagent container, a reader may be provided on the reagent tray, and controller 500 may control the reader to read reagent information from the tag. In such a configuration, the information of each reagent set on the reagent tray is automatically registered in controller 500. The registered reagent information is stored in storage device 530. The reagent information is managed for each reagent ID (thus, for each reagent container).

The analysis device simultaneously performs analyses of a plurality of samples. For example, while preparing for measurement of one sample (for example, dispensation at port P11 or P21), the analysis device performs measurement of another sample (more particularly, optical measurement at optical measurement port P3). In order to perform analyses of all the reserved samples efficiently, the analysis device determines an analysis schedule of each sample based on sample information (for example, a test of analysis of each sample) and a vacancy state of each port, and performs the analyses in accordance with the analysis schedule. The analysis schedule includes, for example, a timing of each of dispensation and measurement, sample and reagent to be dispensed, and optical measurement port P3 at which measurement is to be performed. The analysis schedule is stored in storage device 530. The analysis schedule is managed for each sample ID (thus, for each sample container).

When starting an analysis, an ID (cuvette ID) is provided to a cuvette to be used in the analysis. When the analysis is progressed, an analysis history including an interim progress is stored in storage device 530. The analysis history is sequentially updated in response to progress of the analysis. For example, the analysis history includes: a movement path (inclusive of a current position) of the cuvette; the sample and reagent dispensed to the cuvette; optical measurement port P3 at which the measurement is performed; and a measurement result. The analysis history is managed for each cuvette ID (thus, for each cuvette). By making reference to the analysis history, each of controller 500 and the user can check whether or not the analysis has been performed (or is being progressed) as indicated in the analysis schedule.

While making reference to the analysis schedule and the reagent information, controller 500 controls movable sample rack 32 to place a predetermined sample (i.e., a sample to be dispensed) immediately below port P21, and controls an actuator for driving the turntable of reagent cold storage 31 to place a predetermined reagent (i.e., a reagent to be dispensed) immediately below port P11. The temperature of first probe 11a is adjusted in accordance with a temperature around the analysis device (for example, a temperature of a room in which the analysis device is placed). For example, when the temperature around the analysis device is low, the temperature of first probe 11a is made high. Whenever dispensation is performed using a probe (i.e., first probe 11a or second probe 21a), controller 500 performs control to clean the probe having performed the dispensation.

When a probe is not sufficiently cleaned, accuracy of analysis may be decreased due to an influence of a foreign matter attached on the probe. For example, when a probe having dispensed (previously dispensed) a reagent is not sufficiently cleaned and the probe is then used to dispense (currently dispense) another reagent, contamination (i.e., reagent-to-reagent contamination) may occur due to the previously dispensed reagent. The reagent-to-reagent contamination leads to decreased accuracy of analysis. To address this, the analysis device according to the present embodiment has a below-described configuration to suppress such reagent-to-reagent contamination.

In the analysis device according to the present embodiment, a cleaning condition (hereinafter, referred to as a “first cleaning condition”) for each combination of a reagent previously dispensed by first probe 11a and a reagent currently dispensed by first probe 11a can be set in controller 500. Hereinafter, the combination of the reagent previously dispensed by first probe 11a and the reagent currently dispensed by first probe 11a will be referred to as “reagent combination”. Moreover, a cleaning condition (hereinafter, referred to as “second cleaning condition”) for each combination of a sample previously dispensed by second probe 21a and a sample currently dispensed by second probe 21a can be set in controller 500. Hereinafter, the combination of the sample previously dispensed by second probe 21a and the sample currently dispensed by second probe 21a will be referred to as “specimen combination”. The first cleaning condition and the second cleaning condition correspond to the cleaning condition of first probe 11a and the cleaning condition of second probe 21a, respectively.

The user can set the first cleaning condition and the second cleaning condition in controller 500 through input device 501 (for example, touch panel). When the first cleaning condition and the second cleaning condition are set in controller 500, information (hereinafter, referred to as “probe cleaning information”) indicating the first cleaning condition and the second cleaning condition is stored into storage device 530. Each of the first cleaning condition and the second cleaning condition is a condition of cleaning to be performed during analysis, and is different from a condition of cleaning to be performed when the analysis device is activated (for example, when powered on) and a condition of cleaning to be performed when the analysis device is deactivated (for example, when powered off).

When first probe 11a or second probe 21a has performed dispensation (dispensation corresponding to the “previous dispensation”), controller 500 performs control to clean the probe using a predetermined cleaning liquid (for example, a cleaning liquid indicated by the first cleaning condition or the second cleaning condition) before performing the next dispensation (dispensation corresponding to the “current dispensation”) using the probe. Controller 500 performs control to introduce the cleaning liquid into the probe having performed the dispensation, and discharge the introduced cleaning liquid from the probe, thereby cleaning the probe. Controller 500 can perform control to perform cleaning multiple times by repeating the introduction and discharging of the cleaning liquid. In the present embodiment, pure water and a detergent are employed as the cleaning liquid. Controller 500 performs control to perform cleaning using pure water (hereinafter, also referred to as “pure water cleaning”) and cleaning using a detergent (hereinafter, also referred to as “detergent cleaning”) in below-described manners.

When cleaning a probe (first probe 11a or second probe 21a) using pure water, controller 500 controls a syringe pump (first syringe pump 12 or second syringe pump 22) to supply and introduce, into the probe, pure water having been introduced to the syringe pump and an arm main body (arm main body 11b or 21b) (i.e., pure water supplied in advance from cleaning water tank 40 to the syringe pump and the arm main body by cleaning water pump 43), and discharge the introduced pure water from the probe to a cleaning port (first cleaning port P13 or second cleaning port P23) at a predetermined discharging pressure. Hereinafter, the predetermined discharging pressure will be referred to as “discharging pressure during pure water cleaning”. Thereafter, controller 500 controls a switching valve (first switching valve 41 or second switching valve 42) and cleaning water pump 43 to supply pure water from cleaning water tank 40 to the syringe pump and the arm (arm 11 or 21) to compensate for the insufficiency of pure water due to the discharging.

When cleaning a probe (first probe 11a or second probe 21a) using a detergent, controller 500 controls a syringe pump (first syringe pump 12 or second syringe pump 22) to introduce, into the probe, the detergent provided at detergent port (port P22a, P22b or detergent port P12), by suctioning the detergent into the probe, and to discharge the introduced detergent from the probe to a cleaning port (first cleaning port P13 or second cleaning port P23) at a predetermined discharging pressure after holding it for a predetermined time. Hereinafter, the predetermined time and the predetermined discharging pressure will be referred to as “holding time” and “discharging pressure during detergent cleaning”, respectively. Thereafter, controller 500 controls a switching valve (first switching valve 41 or second switching valve 42) and cleaning water pump 43 to supply pure water from cleaning water tank 40 to the syringe pump and the arm (arm 11 or 21) to wash away the detergent on the probe and introduce pure water to the syringe pump and the arm. The operation of washing away the detergent may be always performed in the same manner or may be changed in accordance with a type of detergent.

Each of the discharging pressure during pure water cleaning and the discharging pressure during detergent cleaning may be variable depending on a situation; however, in the present embodiment, each of the discharging pressures has a fixed value (for example, maximum settable pressure). A discharging amount per discharging corresponds to the discharging pressure. The discharging amount is increased as the discharging pressure is higher.

In the present embodiment, each of the first cleaning condition and second cleaning condition that can be set in controller 500 includes a type of cleaning liquid, a cleaning count, and a holding time. The type of cleaning liquid that can be set in controller 500 include pure water and a detergent. The cleaning count is the number of times of repeating the introduction and discharging of the cleaning liquid. The holding time is a period of time from the suctioning of the detergent by the probe to the discharging of the detergent.

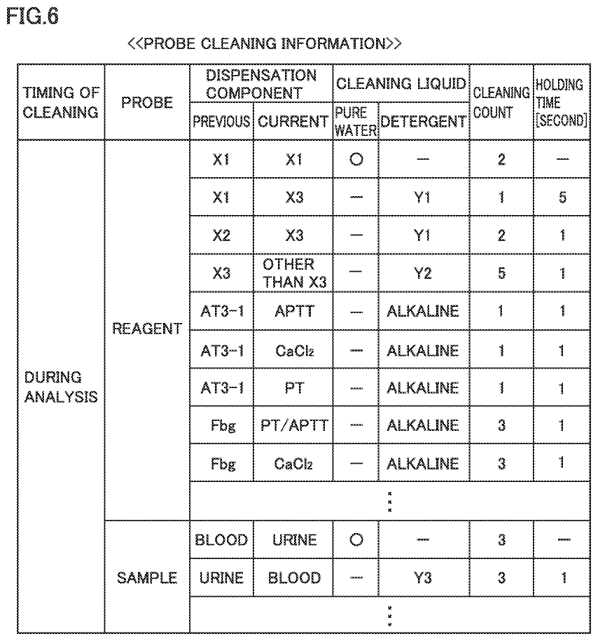

The probe cleaning information in storage device 530 indicates the first cleaning condition and second cleaning condition set in controller 500. FIG. 6 is a diagram showing exemplary probe cleaning information. In FIG. 6, reagents X1, X2, and X3 are different types of reagents, and detergents Y1, Y2, and Y3 are different types of detergents. Hereinafter, a reagent previously dispensed by first probe 11a will be referred to as “previous reagent”, a reagent currently dispensed by first probe 11a will be referred to as “current reagent”, a sample previously dispensed by second probe 21a will be referred to as “previous sample”, and a sample currently dispensed by second probe 21a will be referred to as a “current sample”.

Referring to FIG. 6, this probe cleaning information indicates cleaning conditions as described below. Under a cleaning condition for the combination in which each of the previous reagent and the current reagent is reagent X1, the cleaning liquid is pure water and the cleaning count is two. Under a cleaning condition for the combination in which the previous reagent is reagent X1 and the current reagent is reagent X3, the cleaning liquid is detergent Y1, the cleaning count is one, and the holding time is 5 seconds. Under a cleaning condition for the combination in which the previous reagent is reagent X2 and the current reagent is reagent X3, the cleaning liquid is detergent Y1, the cleaning count is two, and the holding time is 1 second. Under a cleaning condition for the combination in which the previous reagent is reagent X3 and the current reagent is a reagent other than reagent X3, the cleaning liquid is detergent Y2, the cleaning count is five, and the holding time is 1 second.

Under a cleaning condition for the combination in which the previous reagent is AT3 (antithrombin III)-1 and the current reagent is an APTT reagent, the cleaning liquid is an alkaline detergent, the cleaning count is one, and the holding time is 1 second. Under a cleaning condition for the combination in which the previous reagent is AT3-1 and the current reagent is CaCl₂ (calcium chloride), the cleaning liquid is an alkaline detergent, the cleaning count is one, and the holding time is 1 second. Under a cleaning condition for the combination in which the previous reagent is AT3-1 and the current reagent is a PT reagent, the cleaning liquid is an alkaline detergent, the cleaning count is one, and the holding time is 1 second. Under a cleaning condition for the combination in which the previous reagent is Fbg (fibrinogen) and the current reagent is a PT reagent, APTT reagent, or CaCl₂, the cleaning liquid is an alkaline detergent, the cleaning count is three, and the holding time is 1 second. It should be noted that the APTT (activated partial thromboplastin time) reagent is a reagent including an activating substance (foreign matter component) such as an ellagic acid. A PT (prothrombin time) reagent is a reagent including, for example, a tissue factor (TF). AT3-1 refers to a first reagent of two reagents used in measurement for a test of AT3. In two reagents used in measurement for a test of APTT, a first reagent is APTT and a second reagent is CaCl₂. Immediately after dispensation of AT3-1 reagent, the APTT or CaCl₂ is dispensed into the cuvette. When contamination occurs, an ongoing coagulation reaction of the APTT in the cuvette is affected by the AT3-1 reagent.

Under a cleaning condition for the combination in which the previous sample is a blood component (for example, plasma) and the current sample is urine, the cleaning liquid is pure water and the cleaning count is three. Under a cleaning condition for the combination in which the previous sample is urine and the current sample is a blood component (for example, plasma), the cleaning liquid is detergent Y3, the cleaning count is three, and the holding time is 1 second.

Since the detergent has stronger cleaning power than that of the pure water, a matter attached on the probe is more readily removed by the detergent cleaning as compared with the pure water cleaning. Further, as the holding time is longer in the detergent cleaning, the matter attached on the probe tends to be more readily removed. As the cleaning count is increased, a degree of cleanness of the probe tends to be higher. On the other hand, the pure water cleaning can be performed more simply than the detergent cleaning. Further, when the holding time is made long or the cleaning count is increased, the cleaning process becomes complicated or the cleaning time becomes long. Therefore, in the analysis device according to the present embodiment, the cleaning liquid, the cleaning count, and the holding time are changed in accordance with a reagent combination and a specimen combination.

The probe cleaning information shown in FIG. 6 indicates that the probe is cleaned using pure water when the previous reagent and the current reagent are the same (i.e., when reagent-to-reagent contamination is unlikely to occur), whereas the probe is cleaned using a detergent when the previous reagent and the current reagent are different (i.e., when reagent-to-reagent contamination is likely to occur). Uncleanness in the probe due to plasma is readily removed by pure water. Hence, when the previous sample is plasma and the current sample is urine, the probe is cleaned using pure water. In the probe cleaning information shown in FIG. 6, the cleaning condition (more specifically, the cleaning liquid, the cleaning count, and the holding time) is changed in accordance with a reagent combination and a specimen combination, thereby optimizing the degree of cleanness. This makes it possible to suppress contamination of the probe while suppressing excessive cleaning.

When controller 500 obtains a reagent combination and the first cleaning condition is set for the obtained reagent combination, controller 500 performs control to clean first probe 11a under the set first cleaning condition. Further, when controller 500 obtains a specimen combination and the second cleaning condition is set for the obtained specimen combination, controller 500 performs control to clean second probe 21a under the set second cleaning condition.

When no cleaning condition (the first cleaning condition or the second cleaning condition) is set for the obtained combination (the reagent combination or the specimen combination), controller 500 performs control to clean the probe (first probe 11a or second probe 21a) under a predetermined standard condition. Different standard conditions may be set for the cleaning of first probe 11a and the cleaning of second probe 21a, or a common standard condition may be set therefor. In the present embodiment, the common standard condition is employed. The standard condition is stored in storage device 530. The standard condition can be set appropriately. In the standard condition according to the present embodiment, the cleaning liquid is pure water and the cleaning count is one.

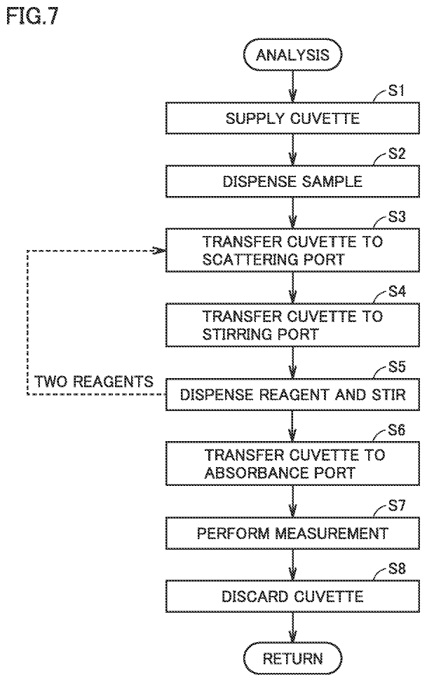

FIG. 7 is a flowchart showing a series of flows of analysis performed by the analysis device according to the present embodiment. Controller 500 performs an analysis in accordance with the above-described analysis schedule. It should be noted that the above-described analysis history is updated whenever an advance is made in the steps of the process shown in FIG. 7.

Referring mainly to FIGS. 3 and 7, in a step (hereinafter, simply referred to as “S”) 1, supplying mechanism 112 takes out cuvettes 100 from accommodation portion 111 and supplies one cuvette 100 to port P1. Based on an output of the port sensor of port P1, supplying mechanism 112 supplies next cuvette 100 to port P1 when port P1 becomes vacant.

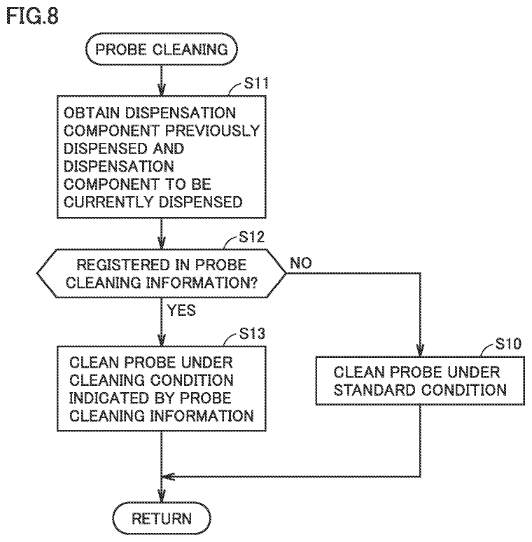

In S2, a sample is dispensed into cuvette 100. More specifically, controller 500 controls movable sample rack 32 (FIG. 2) to place a predetermined sample (more specifically, a sample designated by the analysis schedule) immediately below port P21. Subsequently, controller 500 controls second driving device 23 (FIG. 2) to move second probe 21a to port P21 so as to suction the sample using second probe 21a. Subsequently, controller 500 controls second driving device 23 (FIG. 2) to move second probe 21a to port P1 so as to dispense the sample from second probe 21a to cuvette 100 (more specifically, cuvette 100 supplied to port P1 in S1). After the dispensation, second probe 21a is cleaned by a process shown in FIG. 8 described later.

In S3, cuvette 100 is transferred to optical measurement port P3 for the sake of application of heat. Specifically, controller 500 controls driving device 122 (FIG. 1) to transfer cuvette 100 from port P1 to optical measurement port P3 using arm 121.

In S4, cuvette 100 is transferred to stirring port P2. Specifically, controller 500 controls driving device 122 (FIG. 1) to transfer cuvette 100 from optical measurement port P3 to stirring port P2 using arm 121. It should be noted that when a test of analysis is a test of coagulation, S4 and S6 described later are omitted. In this case, in S5 described later, dispensation is performed into cuvette 100 located at optical measurement port P3, and stirring after the dispensation is not performed. The content of cuvette 100 is mixed due to momentum of discharging the reagent in S5.

In S5, the reagent is dispensed. More specifically, controller 500 controls the actuator for driving the turntable of reagent cold storage 31 to place the predetermined reagent (more specifically, the reagent designated by the analysis schedule) immediately below port P11. Subsequently, controller 500 controls first driving device 13 (FIG. 2) to move first probe 11a to port P-11 so as to suction the reagent using first probe 11a. Subsequently, controller 500 controls first driving device 13 (FIG. 2) to move first probe 11a to stirring port P2 so as to dispense the reagent from first probe 11a to cuvette 100. After the dispensation, the content of cuvette 100 is stirred by stirring device 200. Moreover, after the dispensation, first probe 11a is cleaned by a process of FIG. 8 described later.

When the dispensation of the reagent is finished, cuvette 100 is transferred to optical measurement port P3 in S6. When the test of analysis is a test of absorbance in two reagents, the processes of S3 to S5 are repeated to dispense the first reagent and the second reagent. For example, in S3, cuvette 100 is placed and heated in scattering port P3a. When the dispensation of all the reagents is finished, cuvette 100 is transferred to absorbance port P3b in S6. Thereafter, in S7, controller 500 controls measurement device 300 to perform the measurement described below.

For example, when the sample is plasma and the test of analysis is a test of coagulation, the coagulation time of the sample is measured at scattering port P3a. The intensity of the scattered light is increased in response to progress of coagulation. When the coagulation reaction is ended, the intensity of the scattered light is hardly changed. Hence, the coagulation time can be determined based on the intensity of the scattered light.

When the sample is plasma and the test of analysis is a test of absorbance, the concentration and activity value of the sample are measured at absorbance port P3b. After a predetermined time elapses from the introduction of the sample into cuvette 100, the first reagent is dispensed into cuvette 100. After a predetermined time elapses from the dispensation of the first reagent, the second reagent (more specifically, a reagent different from the first reagent) is dispensed into cuvette 100. By introducing the second reagent into cuvette 100, a reaction between the sample and the reagent is started to change an absorbance of the content of cuvette 100. The concentration and activity value of the sample can be determined based on such a transition of the absorbance. In such a measurement, after dispensing each of the first reagent and the second reagent, first probe 11a is cleaned by the process of FIG. 8 described later.

When the sample is urine, a change in absorbance caused by the reaction between the sample and the reagent is optically measured at absorbance port P3b, for example.

When the above measurement is finished, in S8, controller 500 controls driving device 122 (FIG. 1) to transfer cuvette 100 from optical measurement port P3 to discarding port P4 using arm 121, and to disengage the chuck of arm 121 to introduce cuvette 100 into discarding port P4. When cuvette 100 is introduced into discarding port P4, cuvette 100 (i.e., used reaction container) is collected in discarding container 400 (FIG. 1).

FIG. 8 is a flowchart showing control associated with cleaning of each of first probe 11a and second probe 21a. When dispensation is performed by one of first probe 11a and second probe 21a, the process of FIG. 8 is performed by controller 500.

Referring mainly to FIG. 8, in S11, controller 500 makes reference to an analysis schedule in storage device 530 to obtain a combination of a component previously dispensed by the probe (i.e., a component dispensed immediately before) and a component to be currently dispensed by the probe (i.e., a component to be dispensed next). For example, in the cleaning of first probe 11a, controller 500 obtains a reagent combination. In the cleaning of second probe 21a, controller 500 obtains a specimen combination. Controller 500 may obtain a previously dispensed component (a previous reagent or previous sample) from the analysis history.

In S12, controller 500 makes reference to probe cleaning information (FIG. 5) in storage device 530 and determines whether or not the combination obtained in S11 is registered in the probe cleaning information.

When the combination obtained in S11 is registered in the probe cleaning information (YES in S12), controller 500 performs control to clean the probe (first probe 11a or second probe 21a) under a cleaning condition indicated by the probe cleaning information in S13. More specifically, the above-described pure water cleaning or detergent cleaning is performed. The fact that the combination obtained in S11 is registered in the probe cleaning information means that the cleaning condition (the first cleaning condition or the second cleaning condition) is set in advance for the combination (the reagent combination or the specimen combination) obtained in S11.

On the other hand, when the combination obtained in S11 is not registered in the probe cleaning information (NO in S12), in S10, controller 500 performs control to clean the probe (first probe 11a or second probe 21a) under the standard condition (FIG. 5) in storage device 530. More specifically, for example, the pure water cleaning is performed once. The fact that the combination obtained in S11 is not registered in the probe cleaning information means that the cleaning condition (the first cleaning condition or the second cleaning condition) is not set in advance for the combination (the reagent combination or the specimen combination) obtained in S11.

S11, S12, and S13 according to the present embodiment correspond to examples of “first step”, “second step”, and “third step” according to the present disclosure, respectively.

### Modifications

In the above-described embodiment, prior to cleaning the probe (first probe 11a or second probe 21a), controller 500 obtains the reagent combination or the specimen combination (S11 in FIG. 8). When no cleaning condition of the probe is set for the obtained combination (NO in S12 in FIG. 8), the probe is cleaned under the predetermined standard condition (S10 in FIG. 8). However, it is not limited thereto. When no cleaning condition of the probe is set for the obtained combination (reagent combination or specimen combination), controller 500 may request the user to set a cleaning condition.

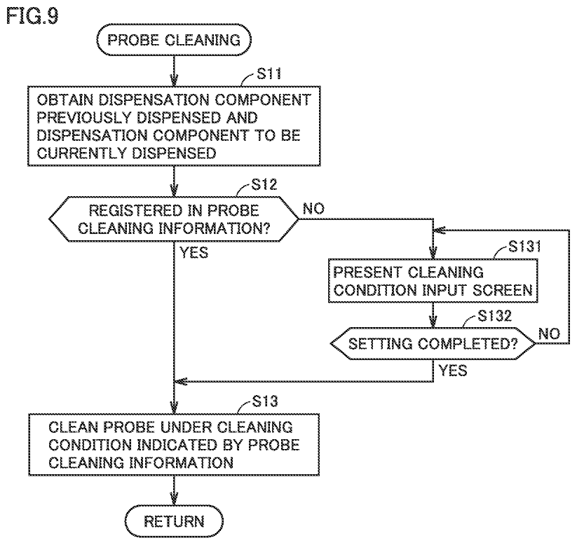

FIG. 9 is a flowchart showing a modification of the control associated with the probe cleaning shown in FIG. 8. Controller 500 may perform a process of FIG. 9 instead of the process of FIG. 8. It should be noted that S11 to S13 in FIG. 9 are the same as S11 to S13 in FIG. 8, respectively.

Referring to FIG. 9 together with FIG. 5, in this modification, when the combination obtained in S11 is not registered in the probe cleaning information (NO in S12), controller 500 requests the user to set a cleaning condition by presenting a cleaning condition input screen on notification device 504 (for example, touch panel display) in S131. In S132, controller 500 determines whether or not a cleaning condition has been set by the user. S131 and S132 are repeated during a period of time until a cleaning condition is set by the user (a period of time during which NO is determined in S132).

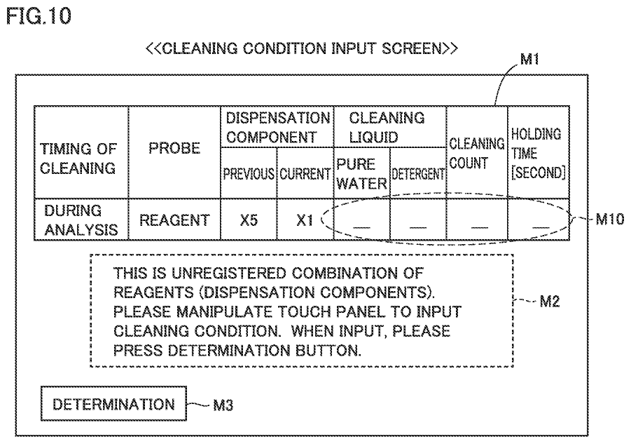

FIG. 10 is a diagram showing an exemplary cleaning condition input screen. Referring to FIG. 10, the cleaning condition input screen presents a table M1, a message M2, and a determination button M3. Table M1 indicates: a reagent combination (previous reagent: reagent X5; current reagent: reagent X1) not set in controller 500; and a cleaning condition (input test M10) that can be set in controller 500. Message M2 includes an explanation regarding the cleaning condition input screen and a message that urges the user to set a cleaning condition. The user can set a cleaning condition in controller 500 by manipulating input device 501 (for example, touch panel). For example, when the user touches a test (input test M10) of the cleaning condition in table M1 on the cleaning condition input screen, options of cleaning conditions or a keypad is presented on the screen, whereby the user can input a cleaning condition. When determination button M3 is pressed by the user after inputting the cleaning condition, the input cleaning condition is set in controller 500 (and is thus registered in the probe cleaning information) and YES is determined in S132 of FIG. 9. Thereafter, in S13 of FIG. 9, the probe is cleaned under the cleaning condition set as described above.

It should be noted that when no cleaning condition of the probe is set for the reagent combination or the specimen combination, controller 500 may be able to perform both of: a first mode (for example, a mode in which the process of FIG. 8 is performed) in which the probe is cleaned under a predetermined standard condition; and a second mode (for example, a mode in which the process of FIG. 9 is performed) in which the user is requested to set a cleaning condition. Controller 500 may perform a mode set by the user in the first and second modes. Such a controller 500 receives an input from the user and performs one of the first mode and the second mode in response to the input (i.e., mode setting) from the user. The user can switch between the modes as required, to cause controller 500 to perform a mode suitable for a situation.

Controller 500 may make reference to the analysis schedule before starting the analysis, and may request the user to set a cleaning condition by presenting a cleaning condition input screen (see, for example, FIG. 10) before starting the analysis, when it is scheduled to perform dispensation of a reagent combination or specimen combination for which no cleaning condition is set in controller 500 during the analysis.

The probe cleaning condition that can be set in controller 500 is not limited to the condition shown in FIG. 6, and may be changed appropriately. For example, the holding time may be set to a fixed value so as to avoid the holding time from being included in the cleaning condition that can be set. Further, cleaning may be performed under a more complex condition with the pure water cleaning and the detergent cleaning being appropriately combined in, for example, the following manner: first detergent cleaning is performed, then pure water cleaning is performed, and second detergent cleaning (for example, cleaning with a detergent different from that in the first detergent cleaning) is then performed.

The structures of the devices (for example, cuvette supplying device 110, cuvette transferring device 120, measurement device 300, and dispensation devices 10, 20) of the blood coagulation analysis device and the arrangements of the ports are not limited to the structures and arrangements shown in FIGS. 3 and 4, and can be changed appropriately. For example, CTS mechanism 24 may be omitted appropriately. Further, in order to suppress introduction of a foreign matter into a port, at least one of the reagent port, the specimen port, and the detergent port may be provided with an openable and closable cover (for example, a slide-type cover), and the cover may be opened only when used.

Any number of probes (dispensation nozzles) may be provided. One arm (for example, arm 11) may have two or more nozzles (first nozzles) that each dispense a reagent. One arm (for example, arm 21) may have two or more nozzles (second nozzles) that each dispense a specimen. In such a configuration in which one arm has two or more dispensation nozzles, information is managed for each dispensation nozzle, and a cleaning condition is set for each dispensation nozzle.

The number of ports can also be changed appropriately. For example, different cleaning ports (first cleaning port P13 and second cleaning port P23) are prepared for the first nozzle and the second nozzle in the above-described embodiment; however, one common cleaning port may be prepared for the first nozzle and the second nozzle. In the above-described embodiment, the plurality of optical measurement ports P3 are prepared in the analysis table; however, one optical measurement port P3 may be provided. The blood coagulation analysis device may sequentially transfer, to optical measurement ports P3, reaction containers for each of which preparation for measurement has been completed. The arm that transfers a reaction container (for example, chuck-equipped arm 121) may be omitted, and the reaction container may be transferred by a belt conveyor system. Each of the reaction containers is not limited to a disposable cuvette composed of acrylic, and any container may be employed as the reaction container.

In the above-described embodiment, the blood coagulation analysis device that analyzes a plurality of types of specimens (blood component and urine) has been illustrated; however, the blood coagulation analysis device may analyze only one type of specimen (for example, blood component).

### ASPECTS

It will be appreciated by one having ordinary skill in the art that the above-described exemplary embodiments and modifications are specific examples of the following aspects.

(Clause 1) A blood coagulation analysis device according to one aspect performs an analysis by reacting a specimen and a reagent in a reaction container, and includes a reagent port, a first dispensation device, and a controller. At the reagent port, a plurality of types of reagents are able to be provided. The first dispensation device has a first nozzle and a first driving device. The first driving device moves the first nozzle to change a position of the first nozzle. The first dispensation device suctions a predetermined reagent of the plurality of types of reagents at the reagent port using the first nozzle, and dispenses the suctioned reagent from the first nozzle to the reaction container. The controller controls the first dispensation device. The controller controls the first dispensation device to dispense the reagent using the first nozzle and to clean the first nozzle whenever the dispensation of reagent is performed. A cleaning condition of the first nozzle is able to be set for each reagent combination in the controller, the reagent combination indicating a combination of a reagent previously dispensed by the first nozzle and a reagent currently dispensed by the first nozzle. When the controller obtains the reagent combination and the cleaning condition of the first nozzle is set for the obtained reagent combination, the controller controls the first dispensation device to clean the first nozzle under the set cleaning condition.

According to the blood coagulation analysis device according to clause 1, the first nozzle can be cleaned under the condition set in advance for each reagent combination indicating the combination of the reagent previously dispensed by the first nozzle for dispensing a reagent and the reagent currently dispensed by the first nozzle for dispensing a reagent. Therefore, the first nozzle is cleaned under the condition suitable for each reagent combination, thereby suppressing reagent-to-reagent contamination in the first nozzle.

(Clause 2) In the blood coagulation analysis device according to clause 1, a cleaning condition (hereinafter, referred to as “same-type reagent condition”) for the reagent combination in which the reagent previously dispensed by the first nozzle and the reagent currently dispensed by the first nozzle are the same, and a cleaning condition (hereinafter, referred to as “different-type reagent condition”) for the reagent combination in which the reagent previously dispensed by the first nozzle and the reagent currently dispensed by the first nozzle are different may be set in the controller. A cleaning liquid indicated by the same-type reagent condition may be pure water. A cleaning solution indicated by the different-type reagent condition may be a detergent.

Since the detergent has stronger cleaning power than that of pure water, uncleanness of the first nozzle is more readily removed by the detergent cleaning as compared with the pure water cleaning. On the other hand, the pure water cleaning can be performed more simply than the detergent cleaning. When the reagent previously dispensed and the reagent currently dispensed are the same, reagent-to-reagent contamination (i.e., contamination by the reagent previously dispensed) is unlikely to occur. Hence, it is considered that even with the pure water cleaning, the first nozzle can have a sufficient degree of cleanness. On the other hand, when the reagent previously dispensed is different from the reagent currently dispensed, reagent-to-reagent contamination is likely to occur. In such a case, by performing the detergent cleaning onto the first nozzle, the first nozzle can have a sufficient degree of cleanness more securely. According to the blood coagulation analysis device according to clause 2, the contamination of the first nozzle can be suppressed while suppressing excessive cleaning.

(Clause 3) In the blood coagulation analysis device according to clause 1 or 2, the controller may control the first dispensation device to clean the first nozzle by introducing a cleaning liquid to the first nozzle having dispensed and discharging the introduced cleaning liquid from the first nozzle. The cleaning condition of the first nozzle may include a type of the cleaning liquid and a cleaning count indicating a number of times of repeating the introduction and the discharging of the cleaning liquid. Each of pure water and a detergent may be able to be set in the controller as the type of the cleaning liquid.

A matter attached on the first nozzle is more readily removed by the detergent cleaning as compared with the pure water cleaning. The degree of cleanness of the first nozzle tends to be higher as the cleaning count of the first nozzle is increased. On the other hand, when the detergent cleaning is performed, the detergent needs to be washed away after finishing the detergent cleaning. When the cleaning count of the first nozzle is increased, the cleaning process becomes complicated or the cleaning time becomes long. According to the blood coagulation analysis device according to clause 3, the degree of cleanness can be optimized for each reagent combination by using either of the pure water and the detergent in accordance with the reagent combination or by changing the cleaning count in accordance with the reagent combination. This makes it possible to suppress contamination of the first nozzle while suppressing excessive cleaning.

(Clause 4) In the blood coagulation analysis device according to clause 3, in the controller, the following cleaning condition may be set for at least one of the following first to sixth combinations. In the first combination, the reagent previously dispensed is AT3-1 and the reagent currently dispensed is an APTT reagent, and in the cleaning condition for the first combination, the cleaning liquid is an alkaline detergent, and the cleaning count is more than or equal to one. In the second combination, the reagent previously dispensed is AT3-1 and the reagent currently dispensed is CaCl₂, and in the cleaning condition for the second combination, the cleaning liquid is an alkaline detergent, and the cleaning count is more than or equal to one. In the third combination, the reagent previously dispensed is AT3-1 and the reagent currently dispensed is a PT reagent, and in the cleaning condition for the third combination, the cleaning liquid is an alkaline detergent, and the cleaning count is more than or equal to one. In the fourth combination, the reagent previously dispensed is Fbg and the reagent currently dispensed is a PT reagent, and in the cleaning condition of the fourth combination, the cleaning liquid is an alkaline detergent, and the cleaning count is more than or equal to three. In the fifth combination, the reagent previously dispensed is Fbg and the reagent currently dispensed is an APTT reagent, and in the cleaning condition for the fifth combination, the cleaning liquid is an alkaline detergent, and the cleaning count is more than or equal to three. In the sixth combination, the reagent previously dispensed is Fbg and the reagent currently dispensed is CaCl₂, and in the cleaning condition of the sixth combination, the cleaning liquid is an alkaline detergent, and the cleaning count is more than or equal to three.

According to the blood coagulation analysis device according to clause 4, the first nozzle can be under a condition suitable for each reagent combination.

(Clause 5) The blood coagulation analysis device according to any one of clauses 1 to 4 may further include a cleaning water tank that accommodates pure water; a cleaning water pump that is controlled by the controller to pump up the pure water from the cleaning water tank; a detergent port at which a detergent is able to be provided; and a cleaning port at which a cleaning liquid is to be collected. The first dispensation device may further have a first syringe pump located between the cleaning water pump and the first nozzle. The cleaning water pump may supply the first syringe pump and the first nozzle with the pure water accommodated in the cleaning water tank. The first syringe pump may be controlled by the controller to adjust suctioning pressure and discharging pressure of the first nozzle. When cleaning the first nozzle using the pure water, the controller may control the first syringe pump to supply and introduce, into the first nozzle, the pure water supplied from the cleaning water tank to the first syringe pump, and to discharge the introduced pure water from the first nozzle to the cleaning port. When cleaning the first nozzle using the detergent, the controller may control the first syringe pump to introduce, into the first nozzle, the detergent provided at the detergent port, by suctioning the detergent into the first nozzle, and to discharge the introduced detergent from the first nozzle to the cleaning port.

In the blood coagulation analysis device according to clause 5, the pure water is supplied from the cleaning water tank to the first nozzle via the first syringe pump using the cleaning water pump (i.e., a pump that adjusts the pressure of the first nozzle). Since the first nozzle is not brought into contact with the pure water in the cleaning water tank when the pure water cleaning is performed, the pure water in the cleaning water tank can be maintained to have a sufficient degree of cleanness. On the other hand, the detergent is provided at the detergent port and is suctioned into the first nozzle. Therefore, the detergent can be selectively attached to the vicinity of the opening of the first nozzle that is likely to be contaminated (i.e., the opening via which suctioning and discharging are performed). By performing the detergent cleaning in this way, the detergent can be readily removed from the first nozzle after finishing the detergent cleaning. In the blood coagulation analysis device according to clause 5, the cleaning port at which the used cleaning liquid (for example, the pure water and the detergent) is to be collected is prepared in addition to the cleaning water tank and the detergent port each for providing the cleaning liquid. With such a cleaning port being provided, contamination due to the used cleaning liquid (for example, contamination of each or the cleaning water tank and the detergent port) is suppressed. Thus, according to the blood coagulation analysis device according to clause 5, the first nozzle can be cleaned simply while suppressing the contamination of the first nozzle.

(Clause 6) In the blood coagulation analysis device according to any one of clauses 1 to 5, when the controller obtains the reagent combination and the cleaning condition of the first nozzle is not set for the obtained reagent combination, the controller may control the first dispensation device to clean the first nozzle under a predetermined standard condition.

According to the blood coagulation analysis device according to clause 6, when the cleaning condition of the first nozzle is not set for the reagent combination, the first nozzle can be cleaned under the predetermined standard condition even though the user does not set a cleaning condition.

(Clause 7) In the blood coagulation analysis device according to any one of clauses 1 to 5, when the controller obtains the reagent combination and the cleaning condition of the first nozzle is not set for the obtained reagent combination, the controller may request a user to set a cleaning condition.

According to the blood coagulation analysis device according to clause 7, when the cleaning condition of the first nozzle is not set for the reagent combination, the controller requests the user to set the cleaning condition. The user can set the cleaning condition in response to the request from the controller.

(Clause 8) The blood coagulation analysis device according to any one of clauses 1 to 7 may further include a specimen port and a second dispensation device. At the specimen port, a plurality of types of specimens may be able to be provided. The second dispensation device may have a second nozzle and a second driving device to move the second nozzle to change a position of the second nozzle. The second dispensation device may suction a predetermined specimen of the plurality of types of specimens at the specimen port using the second nozzle, and may dispense the suctioned specimen from the second nozzle to the reaction container. The controller may control the second dispensation device to dispense the specimen using the second nozzle and to clean the second nozzle whenever the dispensation of specimen is performed. A cleaning condition of the second nozzle may be able to be set for each specimen combination in the controller, the specimen combination indicating a combination of a specimen previously dispensed by the second nozzle and a specimen currently dispensed by the second nozzle. When the controller obtains the specimen combination and the cleaning condition of the second nozzle is set for the obtained specimen combination, the controller may control the second dispensation device to clean the second nozzle under the set cleaning condition.

According to the blood coagulation analysis device according to clause 8, the second nozzle can be cleaned under the condition set in advance for each specimen combination indicating the combination of the specimen previously dispensed by the second nozzle for dispensing a specimen and the specimen currently dispensed by the second nozzle for dispensing a specimen. Therefore, the second nozzle can be cleaned under a condition suitable for each specimen combination. Thus, sample-to-sample contamination in the second nozzle is suppressed.

(Clause 9) The blood coagulation analysis device according to clause 8 may further include a supplying device, a transferring device, a measurement device, and a discarding port. The supplying device may have an accommodation portion to accommodate a plurality of the reaction containers and may supply a reaction container from the accommodation portion. The transferring device may be controlled by the controller to transfer the reaction container. The measurement device may be controlled by the controller to perform a predetermined measurement on a content of the reaction container. At the discarding port, the reaction container may be collected. The controller may control the first dispensation device and the second dispensation device to dispense the specimen and the reagent into the reaction container supplied from the accommodation portion, may control the measurement device to perform the predetermined measurement, and may control the transferring device to discard, to the discarding port, the reaction container having been through the predetermined measurement.

When the specimen and the reagent are dispensed into the reaction container, the specimen and the reagent react with each other in the reaction container to generate a reaction product. It is difficult to completely remove such a foreign matter attached on the reaction container by automatic cleaning. In this regard, in the blood coagulation analysis device according to clause 9, a disposable reaction container is employed. The above-described dispensation and measurement are performed onto a new reaction container (i.e., an unused reaction container) supplied from the accommodation portion of the supplying device, and the reaction container having been through the measurement is then discarded at the discarding port. By performing the above-described dispensation and measurement using a new reaction container having a high degree of cleanness, accuracy of analysis can be improved.

(Clause 10) A method of cleaning a dispensation nozzle according to one aspect is a method of cleaning a dispensation nozzle that dispenses a reagent in a blood coagulation analysis device for performing an analysis by reacting a specimen and the reagent in a reaction container, and includes the following first to third steps. In the first step, a controller of the blood coagulation analysis device obtains a reagent combination indicating a combination of a reagent previously dispensed by the dispensation nozzle and a reagent currently dispensed by the dispensation nozzle. In the second step, the controller determines whether or not a cleaning condition of the dispensation nozzle is set for the reagent combination obtained in the first step. In the third step, when it is determined in the second step that the cleaning condition of the dispensation nozzle is set for the reagent combination, the controller performs control to clean the dispensation nozzle under the cleaning condition set for the reagent combination.

According to the method of cleaning the dispensation nozzle according to clause 10, the dispensation nozzle can be cleaned under the condition set in advance for each reagent combination indicating the combination of the reagent previously dispensed by the dispensation nozzle for dispensing a reagent and the reagent currently dispensed by the dispensation nozzle for dispensing a reagent. Therefore, the dispensation nozzle is cleaned under a condition suitable for each reagent combination, thereby suppressing reagent-to-reagent contamination in the dispensation nozzle.

Although the present invention has been described and illustrated in detail, it is clearly understood that the same is by way of illustration and example only and is not to be taken by way of limitation, the scope of the present invention being interpreted by the terms of the appended claims.

What is claimed is:

1. A blood coagulation analysis device for performing an analysis by reacting a specimen and a reagent in a reaction container, the blood coagulation analysis device comprising:
a reagent port at which a plurality of types of reagents are able to be provided;
a first dispensation device that has a first nozzle and a first driving device to move the first nozzle to change a position of the first nozzle; and
a controller that controls the first dispensation device, wherein
the first dispensation device suctions a predetermined reagent of the plurality of types of reagents at the reagent port using the first nozzle, and dispenses the suctioned reagent from the first nozzle to the reaction container,
the controller controls the first dispensation device to dispense the reagent using the first nozzle and to clean the first nozzle whenever the dispensation of reagent is performed,
in the controller, a cleaning condition of the first nozzle is able to be set for each reagent combination, the reagent combination indicating a combination of a reagent previously dispensed by the first nozzle and a reagent currently dispensed by the first nozzle, and
when the controller obtains the reagent combination and the cleaning condition of the first nozzle is set for the obtained reagent combination, the controller controls the first dispensation device to clean the first nozzle under the set cleaning condition.

2. The blood coagulation analysis device according to claim 1, wherein
a same-type reagent condition and a different-type reagent condition are set in the controller, the same-type reagent condition being a cleaning condition for the reagent combination in which the reagent previously dispensed by the first nozzle and the reagent currently dispensed by the first nozzle are the same, the different-type reagent condition being a cleaning condition for the reagent combination in which the reagent previously dispensed by the first nozzle and the reagent currently dispensed by the first nozzle are different,
a cleaning liquid indicated by the same-type reagent condition is pure water, and
a cleaning liquid indicated by the different-type reagent condition is a detergent.

3. The blood coagulation analysis device according to claim 1, wherein
the controller controls the first dispensation device to clean the first nozzle by introducing a cleaning liquid to the first nozzle having dispensed and discharging the introduced cleaning liquid from the first nozzle,
the cleaning condition of the first nozzle includes
a type of the cleaning liquid and
a cleaning count indicating a number of times of repeating the introduction and the discharging of the cleaning liquid, and
in the controller, each of pure water and a detergent is able to be set as the type of the cleaning liquid.

4. The blood coagulation analysis device according to claim 3, wherein
in the controller, the cleaning condition is set for at least one of
a first combination in which the reagent previously dispensed is AT3-1 and the reagent currently dispensed is an APTT reagent,
a second combination in which the reagent previously dispensed is AT3-1 and the reagent currently dispensed is CaCl₂,
a third combination in which the reagent previously dispensed is AT3-1 and the reagent currently dispensed is a PT reagent,
a fourth combination in which the reagent previously dispensed is Fbg and the reagent currently dispensed is a PT reagent,
a fifth combination in which the reagent previously dispensed is Fbg and the reagent currently dispensed is an APTT reagent, and
a sixth combination in which the reagent previously dispensed is Fbg and the reagent currently dispensed is CaCl₂,
in the cleaning condition for the first combination, the cleaning liquid is an alkaline detergent, and the cleaning count is more than or equal to one,
in the cleaning condition for the second combination, the cleaning liquid is an alkaline detergent, and the cleaning count is more than or equal to one,
in the cleaning condition for the third combination, the cleaning liquid is an alkaline detergent, and the cleaning count is more than or equal to one,
in the cleaning condition for the fourth combination, the cleaning liquid is an alkaline detergent, and the cleaning count is more than or equal to three,
in the cleaning condition for the fifth combination, the cleaning liquid is an alkaline detergent, and the cleaning count is more than or equal to three, and
in the cleaning condition for the sixth combination, the cleaning liquid is an alkaline detergent, and the cleaning count is more than or equal to three.

5. The blood coagulation analysis device according to claim 1, further comprising:
a cleaning water tank that accommodates pure water;
a cleaning water pump that is controlled by the controller to pump up the pure water from the cleaning water tank;
a detergent port at which a detergent is able to be provided; and
a cleaning port at which a cleaning liquid is to be collected, wherein
the first dispensation device further has a first syringe pump located between the cleaning water pump and the first nozzle,
the cleaning water pump supplies the first syringe pump and the first nozzle with the pure water accommodated in the cleaning water tank,
the first syringe pump is controlled by the controller to adjust suctioning pressure and discharging pressure of the first nozzle,
when cleaning the first nozzle using the pure water, the controller controls the first syringe pump to supply and introduce, into the first nozzle, the pure water supplied from the cleaning water tank to the first syringe pump, and to discharge the introduced pure water from the first nozzle to the cleaning port, and
when cleaning the first nozzle using the detergent, the controller controls the first syringe pump to introduce, into the first nozzle, the detergent provided at the detergent port, by suctioning the detergent into the first nozzle, and to discharge the introduced detergent from the first nozzle to the cleaning port.

6. The blood coagulation analysis device according to claim 1, wherein
when the controller obtains the reagent combination and the cleaning condition of the first nozzle is not set for the obtained reagent combination, the controller controls the first dispensation device to clean the first nozzle under a predetermined standard condition.

7. The blood coagulation analysis device according to claim 1, wherein
when the controller obtains the reagent combination and the cleaning condition of the first nozzle is not set for the obtained reagent combination, the controller requests a user to set a cleaning condition.

8. The blood coagulation analysis device according to claim 1, further comprising:
a specimen port at which a plurality of types of specimens are able to be provided; and
a second dispensation device that has a second nozzle and a second driving device to move the second nozzle to change a position of the second nozzle, wherein
the second dispensation device suctions a predetermined specimen of the plurality of types of specimens at the specimen port using the second nozzle, and dispenses the suctioned specimen from the second nozzle to the reaction container
the controller controls the second dispensation device to dispense the specimen using the second nozzle and to clean the second nozzle whenever the dispensation of specimen is performed,
in the controller, a cleaning condition of the second nozzle is able to be set for each specimen combination, the specimen combination indicating a combination of a specimen previously dispensed by the second nozzle and a specimen currently dispensed by the second nozzle, and
when the controller obtains the specimen combination and the cleaning condition of the second nozzle is set for the obtained specimen combination, the controller controls the second dispensation device to clean the second nozzle under the set cleaning condition.

9. The blood coagulation analysis device according to claim 8, further comprising:
a supplying device that has an accommodation portion to accommodate a plurality of the reaction containers and that supplies a reaction container from the accommodation portion;
a transferring device that is controlled by the controller to transfer the reaction container;
a measurement device that is controlled by the controller to perform a predetermined measurement on a content of the reaction container; and
a discarding port at which the reaction container is to be collected, wherein
the controller controls the first dispensation device and the second dispensation device to dispense the specimen and the reagent into the reaction container supplied from the accommodation portion, controls the measurement device to perform the predetermined measurement, and controls the transferring device to discard, to the discarding port, the reaction container having been through the predetermined measurement.

10. A method of cleaning a dispensation nozzle that dispenses a reagent in a blood coagulation analysis device for performing an analysis by reacting a specimen and the reagent in a reaction container, the method comprising:
obtaining, by a controller of the blood coagulation analysis device, a reagent combination indicating a combination of a reagent previously dispensed by the dispensation nozzle and a reagent currently dispensed by the dispensation nozzle;
determining, by the controller, whether or not a cleaning condition of the dispensation nozzle is set for the reagent combination obtained in the obtaining of the reagent combination; and
when it is determined that the cleaning condition of the dispensation nozzle is set for the reagent combination, performing control, by the controller, to clean the dispensation nozzle under the cleaning condition set for the reagent combination.
